(19) United States
(12) Patent Application Publication
(10) Pub. No.: US 2025/0161521 A1
(43) Pub. Date: May 22, 2025

(54) AIR STERILIZATION MODULE, AIR PURIFICATION DEVICE, AND METHOD FOR ASSEMBLING AIR STERILIZATION MODULE

(71) Applicant: Samsung Electronics Co., Ltd., Suwon-si (KR)

(72) Inventors: Changhyun PARK, Suwon-si (KR); Sunghyun CHUN, Suwon-si (KR); Nakhyun KIM, Suwon-si (KR); Juyoung KIM, Suwon-si (KR); Huiyoung KIM, Suwon-si (KR); Juwan PARK, Suwon-si (KR); Changwoo JUNG, Suwon-si (KR); Seoyoung CHO, Suwon-si (KR)

(21) Appl. No.: 19/034,093

(22) Filed: Jan. 22, 2025

Related U.S. Application Data
(63) Continuation of application No. PCT/KR2023/013056, filed on Sep. 1, 2023.

(30) Foreign Application Priority Data
Oct. 6, 2022 (KR) 10-2022-0128135
Nov. 11, 2022 (KR) 10-2022-0150974

(51) Int. Cl. A61L 9/20

(52) U.S. Cl. CPC A61L 9/20; A61L 2209/11; A61L 2209/12

(57) ABSTRACT

An air sterilization module according to an example embodiment includes: a housing including an intake port and an exhaust port, a main body module provided inside the housing and including a fan configured to generate airflow, a display module including a display configured to be exposed to outside of the housing, and a fixing assembly comprising a fastener configured to separably fix the display module to the main body module, wherein the fixing assembly may include a fixing member comprising a guide rail or protrusion fixed to the main body module and a support member comprising a support configured to be assembled to the fixing member while supporting the display module, any one of the fixing member and the support member may include a guide rail extending in a direction in which the support member is assembled, and an other of the fixing member and the support member may include a guide protrusion that is inserted into the guide rail and is movable along the guide rail.

### CROSS-REFERENCE TO RELATED APPLICATIONS

This application is a continuation of International Application No. PCT/KR2023/013056 designating the United States, filed on Sep. 1, 2023, in the Korean Intellectual Property Receiving Office and claiming priority to Korean Patent Application Nos. 10-2022-0128135, filed on Oct. 6, 2022, and 10-2022-0150974, filed on Nov. 11, 2022, in the Korean Intellectual Property Office, the disclosures of each of which are incorporated by reference herein in their entireties.

### BACKGROUND

### Field

The disclosure relates to an air sterilization module, an air purification device, and a method of assembling the air sterilization module.

### Description of Related Art

Air purification devices are used to remove contaminants from air. Air purifiers can remove chemicals, such as bacteria, viruses, fungi, fine dust, and the like, which cause bad odors, from inhaled air.

An air purification device may include an intake port for drawing in polluted air, an exhaust port for discharging purified air, and a blower fan for generating airflow.

The air purification device may include an air sterilization module that sterilizes microorganisms in the air.

The air sterilization module may sterilize microorganisms, such as germs, viruses, and the like in the air by emitting ultraviolet rays into the airflow.

### SUMMARY

An air sterilization module according to an example embodiment may include a housing including an intake port and an exhaust port.

The air sterilization module according to an example embodiment may include a main body module provided inside the housing and comprising a fan configured to generate airflow.

The air sterilization module according to an example embodiment may include a display module comprising a display provided to be visible to the outside of the housing.

The air sterilization module according to an example embodiment may include a fixing assembly comprising a fastener configured to separably fix the display module to the main body module.

In the air sterilization module according to an example embodiment, the fixing assembly comprises a fixing member fixed to the main body module and a support member comprising a support configured to be assembled to the fixing member while supporting the display module, any one of the fixing member and the support member may include a guide rail extending in a direction in which the support member is assembled, and an other of the fixing member and the support member may include a guide protrusion inserted into the guide rail and movable along the guide rail.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a perspective view of an air sterilization module according to various embodiments;

FIG. 2 is an exploded perspective view of the air sterilization module according to various embodiments;

FIG. 3 is an exploded perspective view of a main body module of the air sterilization module, according to various embodiments;

FIG. 4 is a cross-sectional view of the air sterilization module according to various embodiments;

FIG. 5 is an exploded perspective view of a display module, a fixing assembly, and a housing, according to various embodiments;

FIG. 6 is a perspective view of the display module and the fixing assembly, according to various embodiments;

FIG. 7 is an exploded perspective view of the display module and the fixing assembly, according to various embodiments;

FIG. 8 is a perspective view of a support member assembled to the fixing member, according to various embodiments;

FIG. 9 is a perspective view of the support member according to various embodiments;

FIG. 10 is a perspective view of the fixing member according to various embodiments;

FIG. 11 is a perspective view illustrating a locking portion according to various embodiments;

FIG. 12 is a partial perspective view illustrating a stopper according to various embodiments;

FIG. 13 is a perspective view illustrating an accommodation portion according to various embodiments;

FIG. 14 is a partial perspective view illustrating an accommodation groove according to various embodiments;

FIG. 15 is an exploded perspective view of a display housing according to various embodiments;

FIG. 16 is a cross-sectional view of the display housing and the support member, according to various embodiments;

FIG. 17 is an enlarged cross-sectional view of a connection portion of the display module, the fixing assembly, and the main body module of the air sterilization module, according to various embodiments;

FIG. 18 is a flowchart illustrating an example method of assembling the air sterilization module, according to various embodiments;

FIG. 19 is a flowchart illustrating an example operation of assembling the support member to the fixing member, according to various embodiments;

FIG. 20 is a flowchart illustrating an example method of assembling the air sterilization module, according to various embodiments;

FIG. 21 is a flowchart illustrating an example operation of fixing the display module to the support member, according to various embodiments;

FIG. 22 is a flowchart illustrating an example operation of preparing the support member, according to various embodiments;

FIG. 23 is a cross-sectional view illustrating a state of the display module and the fixing assembly, according to various embodiments;

FIG. 24 is a cross-sectional view illustrating a state of the display module and the fixing assembly, according to various embodiments;

FIG. 25 is a perspective view of an air purification device according to various embodiments; and

FIG. 26 is a cross-sectional view of an air purification module according to various embodiments.

### DETAILED DESCRIPTION

Hereinafter, an air sterilization module, an air purification device, and an assembly method thereof, according to various embodiments, are described in greater detail with reference to the accompanying drawings. Throughout the drawings, like reference numerals denote like elements, and sizes of components in the drawings may be exaggerated for convenience of explanation and clarity. Terms such as first, second, etc. may be used for describing various components, but the components may not be limited to those terms. Such terms are used only for the purpose of distinguishing one element from another element.

As used herein, the singular forms “a,” “an,” and “the” are intended to include the plural forms as well, unless the context clearly indicates otherwise. Furthermore, when a part may “include” a certain element, unless specified otherwise, it may not be construed to exclude another element but may be construed to further include other elements. Furthermore, the size or thickness of each element illustrated in the drawings may be exaggerated for clarity of explanation.

The use of terms “a,” “an,” “the,” and similar referents in the context of describing the disclosure are to be construed to cover both the singular and the plural.

The steps of the methods described herein can be performed in any suitable order unless otherwise indicated herein or otherwise clearly contradicted by context. The use of any and all examples, or language (e.g., “such as”) provided herein, is intended merely to better illuminate the disclosure and does not pose a limitation on the scope of the disclosure unless otherwise claimed.

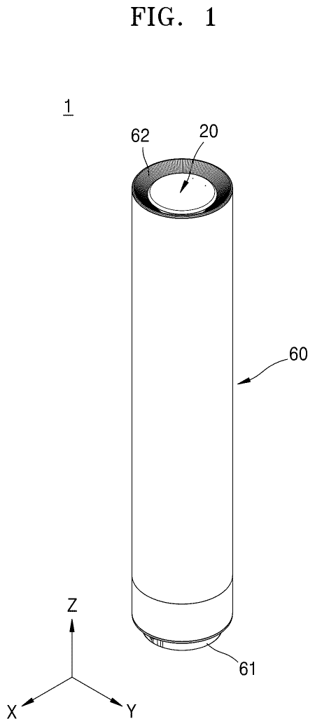

Terms, such as “an up/down direction,” “a height direction,” “a vertical direction,” etc., may refer, for example, to a Z direction based on the perspective view of an air purification device of FIG. 1, “a horizontal direction” may refer, for example, to all directions according to an X-Y plane in an X direction or a Y direction based on the perspective view of FIG. 1, and the shape and the position of each component are not limited by the terms.

The terms, such as “a portion, a module, a member, or a block” used in the disclosure, may be implemented by software or hardware, according to various embodiments, a plurality of “portions, modules, members, or blocks” are implemented by one component, or one “portion, module, member, or block” may include a plurality of components.

In the following description, an air sterilization module according to various example embodiments is described in greater detail with reference to the accompanying drawings.

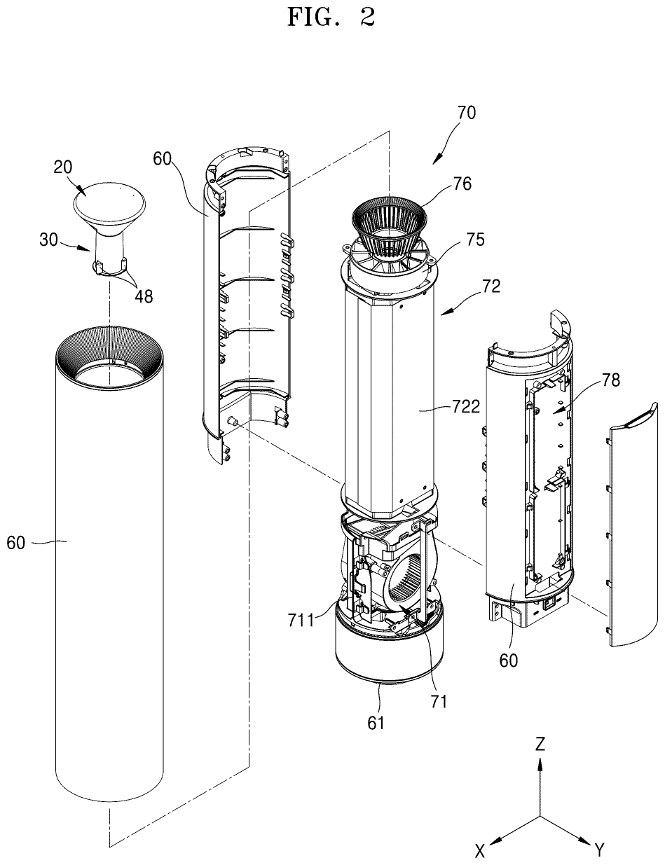

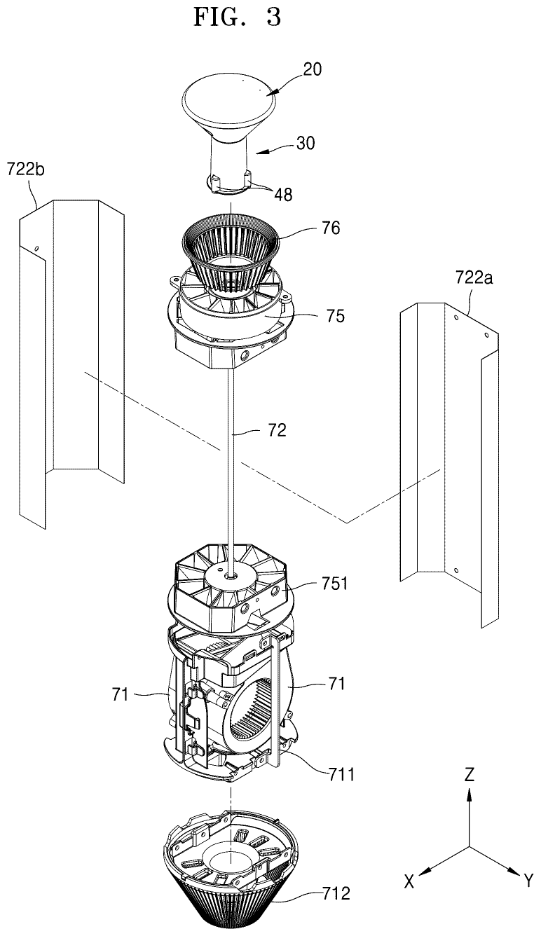

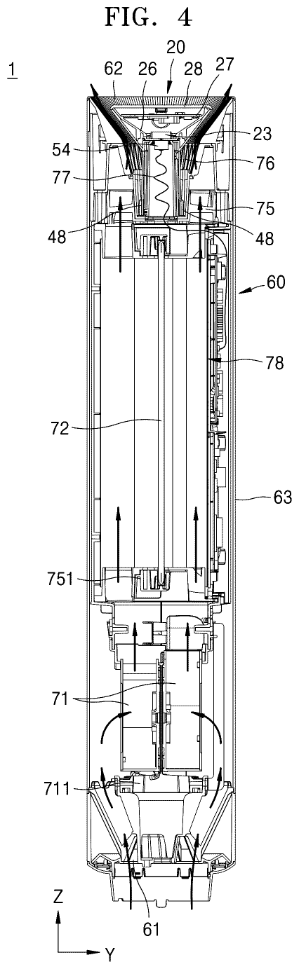

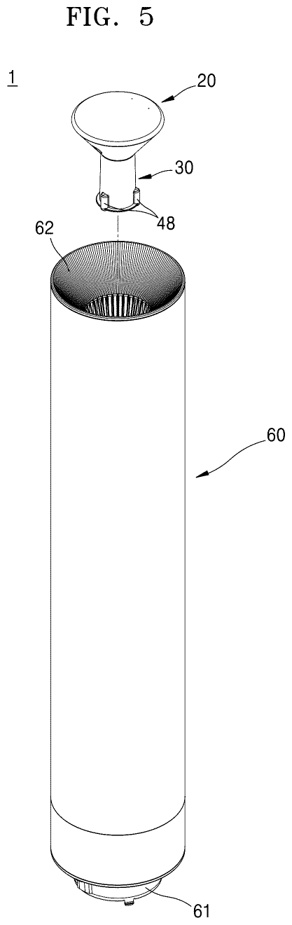

FIG. 1 is a perspective view of an air sterilization module according to various embodiments. FIG. 2 is an exploded perspective view of an air sterilization module according to various embodiments. FIG. 3 is an exploded perspective view of a main body module of an air sterilization module according to various embodiments. FIG. 4 is a cross-sectional view of an air sterilization module according to various embodiments. FIG. 5 is an exploded perspective view of a display module, a fixing assembly, and a housing according to various embodiments.

Referring to FIGS. 1, 2, 3, 4 and 5 (which may be referred to as FIGS. 1 to 5), an air sterilization module 1 according to an example embodiment may be configured to sterilize air introduced from the outside and discharge sterilized air back to the outside.

The air sterilization module 1 according to an example embodiment may include a housing 60, a main body module (e.g., including a main body) 70, a display module (e.g., including a display) 20, and a fixing assembly (e.g., including a fastener) 30.

The housing 60 may form the appearance of the air sterilization module 1. The housing 60 may have a function to accommodate the main body module 70. The housing 60 may accommodate the display module 20 and the fixing assembly 30.

The housing 60 may include an intake port 61 through which external air is drawn in and an exhaust port 62 through which sterilized air is discharged to the outside. The location of the intake port 61 may be in a lower portion of the housing 60, and the location of the exhaust port 62 may be in an upper portion of the housing 60. However, the locations of the intake port 61 and the exhaust port 62 are not limited thereto. The housing 60 may have an approximately cylindrical shape. However, the shape of the housing 60 is not limited thereto.

The main body module 70 may be provided inside the housing 60. The main body module 70 may perform an air sterilization function. The main body module 70 may perform an air purification function. However, the function of the main body module 70 is not limited thereto. For example, the main body module 70 may simultaneously perform the air purification function and the air sterilization function.

The main body module 70 may include a fan 71. The fan 71 may generate airflow inside the housing 60. The main body module 70 may suck air through the intake port 61, sterilize the air, and then discharge the air through the exhaust port 62.

The main body module 70 may include a fan support frame 711 and a fan guard 712. The fan 71 may be supported by the fan support frame 711.

The main body module 70 may include an ultraviolet sterilization module 72 for generating an ultraviolet ray and a reflection cover 722. The ultraviolet sterilization module 72 may sterilize microorganisms in the air by emitting ultraviolet light thereto. The reflection cover 722 may reflect the ultraviolet light therein. The reflection cover 722 may include a first reflection cover 722a and a second reflection cover 722b. The first reflection cover 722a and the second reflection cover 722b may each have an asymmetrical shape. However, the shapes of the first reflection cover 722a and the second reflection cover 722b are not limited thereto.

The main body module 70 may include an upper light blocking member 75 and a lower light blocking member 751. The upper light blocking member 75 may prevent and/or reduce the ultraviolet light emitted from the ultraviolet sterilization module 72 from being discharged through an upper portion of the air sterilization module 1. The lower light blocking member 751 may prevent and/or reduce the ultraviolet light from being discharged through a lower portion of the air sterilization module 1.

The main body module 70 may include a grill top 76. The grill top 76 may include a grill through which air is discharged. The display module 20 may be provided to be exposed to the outside of the housing 60. The display module 20 being exposed from the housing 60 may be the display module 20 accommodated in the housing 60 being popped up to the outside the housing 60. The display module 20 being exposed to the outside of the housing 60 may be that a display cover 28 to be described below is exposed to the outside of the housing 60. The display module 20 being exposed to the outside of the housing 60 may refer, for example, to a user being able to see the display module 20 without disassembling the housing 60. The display module 20 may be disposed in the upper portion of the housing 60. However, the location of the display module 20 is not limited thereto.

The fixing assembly 30 may include a main body fixing portion 48. The main body fixing portion 48 may be fixed to the main body module 70.

The main body fixing portion 48 may be fixed to the upper light blocking member 75.

The fixing assembly 30 may be connected to the display module 20. The fixing assembly 30 may be connected to the main body module 70. The fixing assembly 30 may have a function to fix a fixing member 40 to the main body module 70.

The fixing assembly 30 may include the main body fixing portion 48. The main body fixing portion 48 may have a function to fix the fixing member 40 to the main body module 70. The main body fixing portion 48 may be positioned in a lower portion of the fixing member 40, but the disclosure is not limited thereto. The main body fixing portion 48 may include a plurality of main body fixing members.

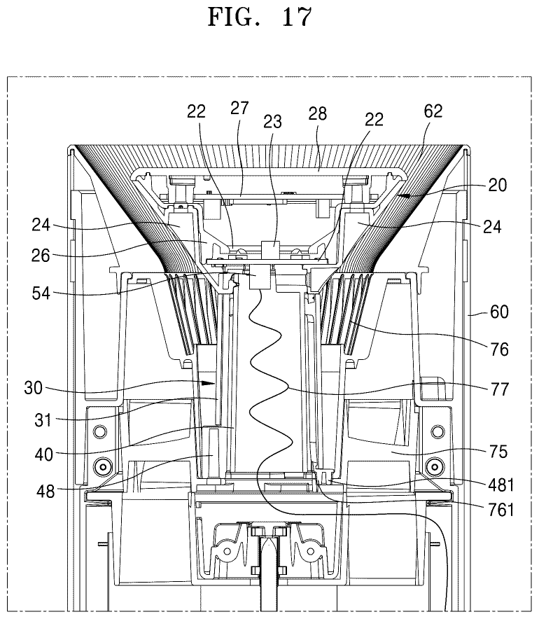

The main body fixing portion 48 may be fixed to the upper light blocking member 75. A fastening member (not shown) that penetrates the main body fixing portion 48 and the upper light blocking member 75 may exist. The fastening member may be screw-coupled to the main body fixing portion 48 by penetrating the upper light blocking member 75. The fixing member 40 may fix the grill top 76 to the upper light blocking member 75. A grill top latch 481 (see FIG. 17) of the fixing member 40 is caught by a grill top catch hole 761 (see FIG. 17) of the grill top 76 so that the grill top 76 may be fixed.

The fixing assembly 30 may serve to fix the display module 20 to the main body module 70. The fixing assembly 30 may serve to assemble the display module 20 to the main body module 70. The fixing assembly 30 may function to fix the display module 20 to the main body module 70 in a separable way. The fixing assembly 30 may have an approximately cylindrical shape. However, the shape of the fixing assembly 30 is not limited thereto.

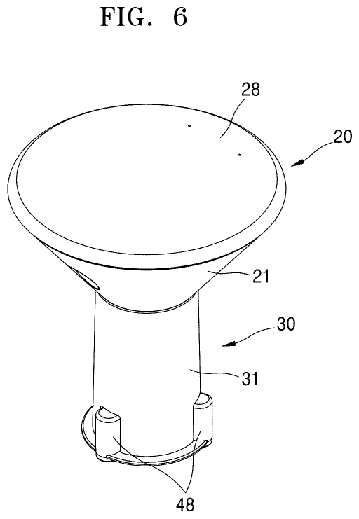

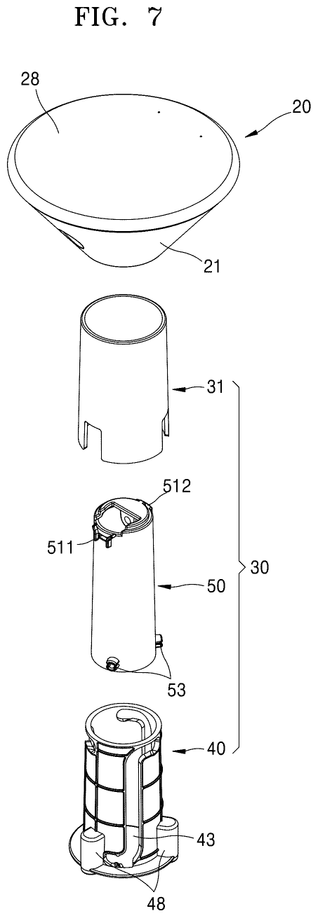

FIG. 6 is a perspective view of the display module and the fixing assembly according to various embodiments. FIG. 7 is an exploded perspective view of the display module and the fixing assembly according to various embodiments.

Referring to FIGS. 6 and 7, the display module 20 according to an example embodiment may be connected to the fixing assembly 30. The display module 20 may be connected to a support member 50.

The display module 20 may perform a display function. The display module 20 may provide the user with information about an operation of the air sterilization module 1. The display module 20 may include the display cover 28. The display cover 28 may form the appearance of the display module 20. The display cover 28 may perform a display function. The display cover 28 may provide the user with information about the operation of the air sterilization module 1. The display cover 28 may include a touch panel. The user may control the operation of the air sterilization module 1 by manipulating the display cover 28. The display cover 28 may be located on top of the display module 20. The display cover 28 may have a circular shape. However, the location or the shape of the display cover 28 is not limited thereto.

The display module 20 may include a display housing 21. The display housing 21 may form the appearance of the display module 20. The display housing 21 may have a function to accommodate parts of the display module 20. The display housing 21 may have a shape in which a cross-sectional area of the display housing 21 increases from the lower portion to the upper portion of the display housing 21. The display housing 21 may have an approximately funnel shape. The shape of the display housing 21 is not limited thereto.

The fixing assembly 30 may include the fixing member 40. The fixing member 40 may have a function to fix the display module 20 to the main body module 70. Referring to FIGS. 2, 3, 4, 6, and 7, the fixing member 40 may be fixed to the upper light blocking member 75. The fastening member may penetrate the fixing member 40 and the upper light blocking member 75 to fix the fixing member 40 to the upper light blocking member 75.

Referring back to FIGS. 6 and 7, the fixing member 40 may have a function to support the support member 50. The fixing member 40 may have a function to fix the support member 50. The fixing member 40 may have a function to accommodate the support member 50. However, the disclosure is not limited thereto, and the support member 50 may accommodate the fixing member 40. The fixing member 40 may have an approximately cylindrical shape. However, the shape of the fixing member 40 is not limited thereto.

The fixing assembly 30 may include the support member 50. The support member 50 may have a function to physically connect the display module 20 to the fixing member 40. The support member 50 may be assembled to the fixing member 40. The support member 50 may support the display module 20. The support member 50 may have an approximately cylindrical shape. However, the shape the support member 50 is not limited thereto.

A guide protection case 31 may have a function to protect the support member 50. The guide protection case 31 may have a function to protect the fixing member 40. The guide protection case 31 may have a function to protect a guide rail 43 of the fixing member 40 which is described below. The guide protection case 31 may have a function to prevent or block the guide rail 43 and a guide protrusion 53 inserted into the guide rail 43 from being exposed to the outside. The guide protection case 31 may accommodate the support member 50. The guide protection case 31 may accommodate the fixing member 40. The guide protection case 31 may surround the fixing member 40. The guide protection case 31 may form the appearance of the fixing assembly 30. The guide protection case 31 may have an approximately cylindrical shape. However, the shape of the guide protection case 31 is not limited thereto.

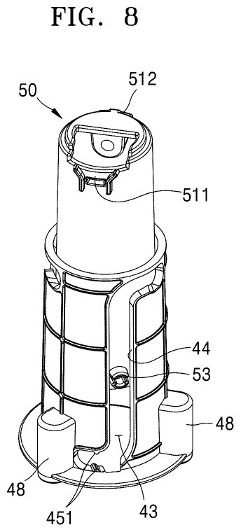

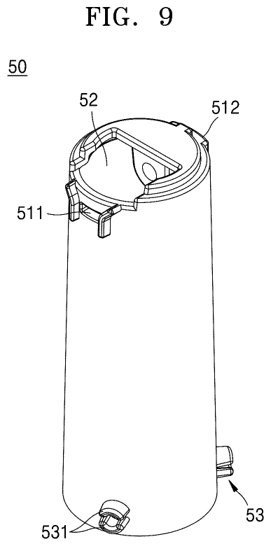

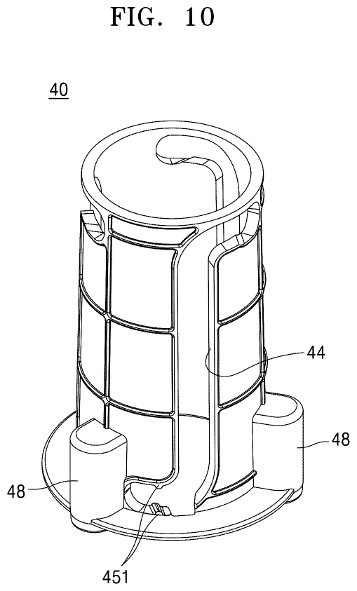

FIG. 8 is a perspective view of the support member assembled to the fixing member according to various embodiments. FIG. 9 is a perspective view of the support member according to various embodiments. FIG. 10 is a perspective view of the fixing member according to various embodiments.

Referring to FIGS. 8, 9 and 10 (which may be referred to as FIGS. 8 to 10), the fixing member 40 according to an example embodiment may include the guide rail 43. The guide rail 43 may have a function to guide a movement of the guide protrusion 53 to be described below. The guide rail 43 may include a guide portion 44.

The guide portion 44 may extend in a direction in which the support member 50 is assembled. The guide portion 44 may extend in a vertical direction. The guide portion 44 may extend in a longitudinal direction of the housing 60. The guide portion 44 may extend at a certain angle in the longitudinal direction of the housing 60. The longitudinal direction may be referred to as a first direction. However, the shape of the guide portion 44 is not limited thereto. The guide rail 43 may include a plurality of guide rails. The guide rail 43 may include three or more guide rails.

The support member 50 according to an example embodiment may include the guide protrusion 53. The guide protrusion 53 may guide a movement of the support member 50. The size of the guide protrusion 53 may be less than the width of the guide rail 43. The guide protrusion 53 may move on the guide rail 43. The guide protrusion 53 may be inserted into the guide rail 43. An end portion of the guide protrusion 53 may be elastically deformable. The guide protrusion 53 may include a plurality of partial protrusions 531. The guide protrusion 53 may include a plurality of guide protrusions. The guide protrusion 53 may include three or more guide protrusions. The number of the guide protrusions 53 may be equal to or less than the number of the guide rails 43.

The diameter of the support member 50 may be less than the inner diameter of the fixing member 40. The support member 50 may accommodate the fixing member 40. The diameter of the support member 50 including the size of the guide protrusion 53 may be greater than the inner diameter of the fixing member 40. The support member 50 may be assembled to the fixing member 40. The support member 50 being assemble to the fixing member 40 may be that the guide protrusion 53 is elastically deformed and arranged on the guide rail 43. The guide protrusion 53 may move on the guide portion 44.

However, the disclosure is not limited thereto, and the inner diameter of the support member 50 may be greater than the outer diameter of the fixing member 40. For example, the guide protrusion 53 may be arranged on the inner circumferential surface of the support member 50. In this case, as the support member 50 is arranged outside the fixing member 40, the support member 50 may function as the guide protection case 31.

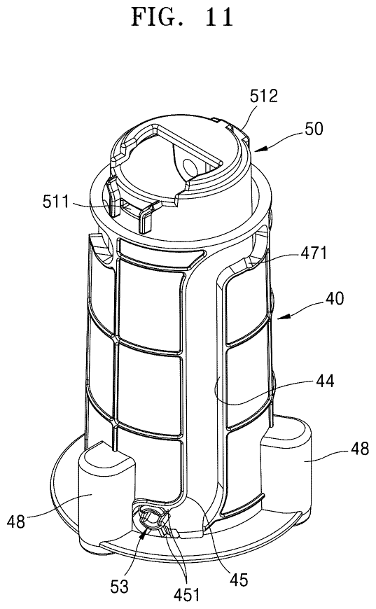

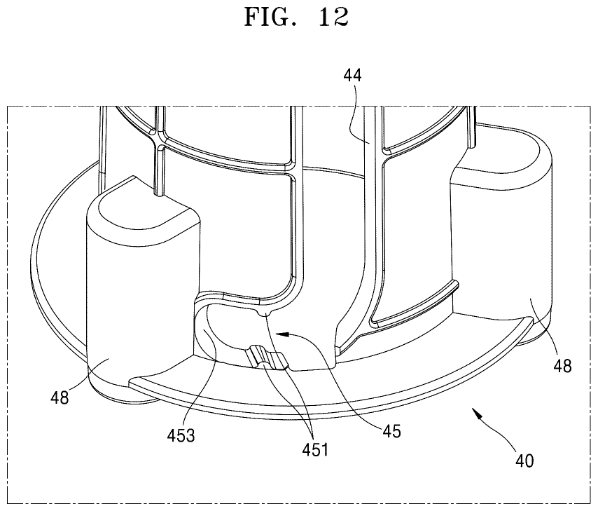

FIG. 11 is a perspective view illustrating a locking portion according to various embodiments. FIG. 12 is a partial perspective view illustrating a stopper according to various embodiments.

Referring to FIGS. 9, 10, 11 and 12 (which may be referred to as FIGS. 9 to 12), the guide rail 43 according to an example embodiment may include a locking portion 45. The locking portion 45 may have a function to fix the support member 50 to the fixing member 40 by rotating the support member 50. The locking portion 45 may be configured to fix the position of the guide protrusion 53. The locking portion 45 may be connected to a lower end of the guide portion 44. The locking portion 45 may extend in a direction intersecting a direction in which the guide portion 44 extends. The direction in which the locking portion 45 extends may be a second direction.

The locking portion 45 may include a stopper 451. The stopper 451 may have a function to fix the position of the guide protrusion 53. The guide protrusion 53 may be elastically deformed while passing the stopper 451. A locking position 453 may be an area in which the guide protrusion 53 is stably accommodated. The guide protrusion 53 may be a protrusion protruding from the locking portion 45. The guide protrusion 53 may include a plurality of guide protrusions.

The guide protrusion 53 may be moved from the guide portion 44 to the locking portion 45 by applying rotational power to the support member 50 relative to the fixing member 40. When the display module 20 is connected to the support member 50, the guide protrusion 53 may be moved from the guide portion 44 to the locking portion 45 by applying rotational power to the display module 20 relative to the fixing member 40.

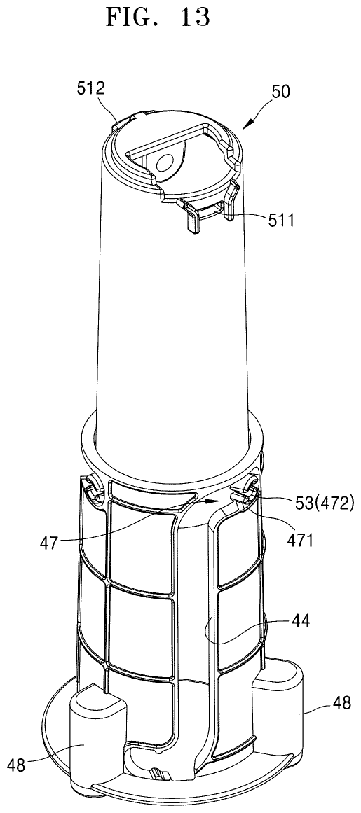

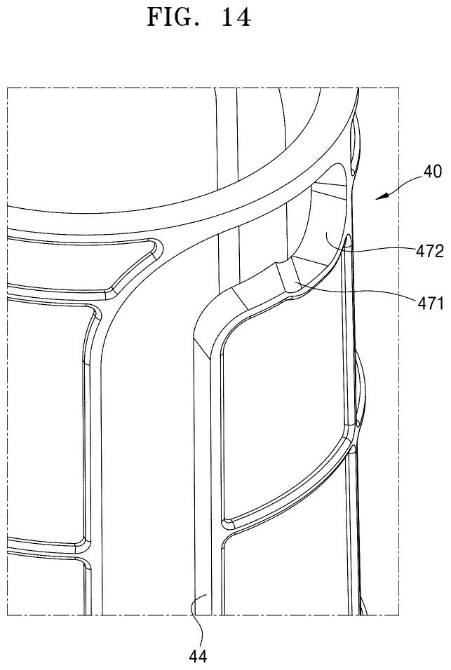

FIG. 13 is a perspective view illustrating an accommodation portion according to various embodiments. FIG. 14 is a partial perspective view illustrating an accommodation groove according to various embodiments.

Referring to FIGS. 9, 10, 13, and 14, the guide rail 43 according to an example embodiment may include an accommodation portion 47. The accommodation portion 47 may have a function to fix the support member 50 to the fixing member 40 by rotating the support member 50. The accommodation portion 47 may have a function to fix the position of the support member 50 in a state of being raised upwards in the fixing member 40. The accommodation portion 47 may facilitate the user to disassemble the display module 20 from the support member 50. The accommodation portion 47 may be connected to an upper end of the guide portion 44. The accommodation portion 47 may extend in a direction intersecting a direction in which the guide portion 44 extends. The direction in which the accommodation portion 47 extends may be a third direction. The third direction may be opposite to the second direction. However, the third direction is not necessarily limited thereto, and may be the same direction as the second direction.

The accommodation portion 47 may include an accommodation groove 471 and an accommodation position 472. The accommodation groove 471 may restrict a movement of the guide protrusion 53 when the guide protrusion 53 is located in the accommodation portion 47. The accommodation groove 471 may have a shape of being recessed in a direction in which the width of the guide portion 44 increases. However, the shape of the accommodation groove 471 is not limited thereto. For example, the accommodation groove 471 may be provided by a stopper 451 (see FIG. 12). When the guide protrusion 53 is located on the accommodation groove 471 in the accommodation portion 47, the guide protrusion 53 may be positioned at the accommodation position 472. When the guide protrusion 53 is located at the accommodation position 472, the display module 20 connected to the support member 50 may be separated.

The guide protrusion 53 may be moved from the guide portion 44 to the accommodation portion 47 by applying rotational power to the support member 50 relative to the fixing member 40. When the display module 20 is connected to the support member 50, the guide protrusion 53 may be moved from the guide portion 44 to the accommodation portion 47 by applying rotational power to the display module 20 relative to the fixing member 40.

Although, in the above descriptions, it is assumed that the fixing member 40 includes the guide rail 43 and the support member 50 includes the guide protrusion 53, the disclosure is not limited thereto. For example, it may be possible that the fixing member 40 includes the guide protrusion 53 and the support member 50 includes the guide rail 43. For example, the guide protrusion 53 may be arranged on an inner circumferential surface of the fixing member 40, or the guide protrusion 53 may be arranged on an outer circumferential surface of the fixing member 40.

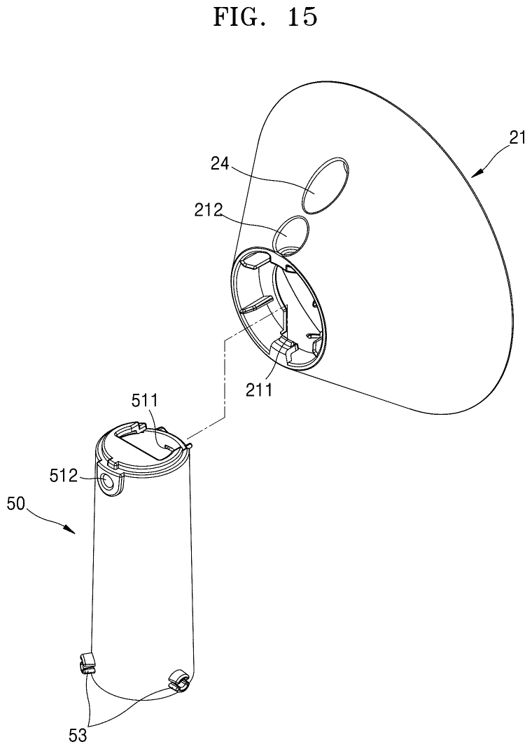

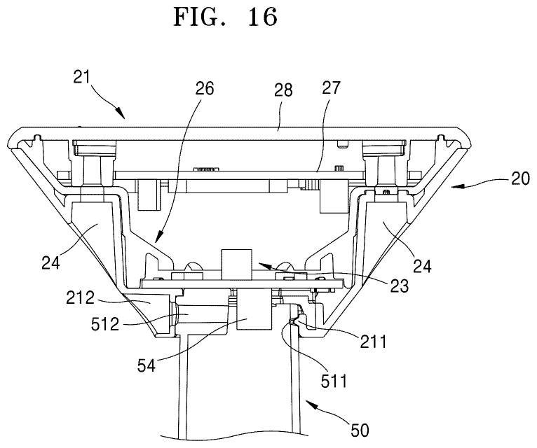

FIG. 15 is an exploded perspective view of the display housing 21 and the support member 50 of according to various embodiments. FIG. 16 is a cross-sectional view of the display housing 21 and the support member 50 according to various embodiments.

Referring to FIGS. 15 and 16, the display housing 21 according to an example embodiment may include a hook 211 and a first fastening hole 212. The hook 211 may have a function to connect the display module 20 to the support member 50. A fastening screw (not shown) inserted into the first fastening hole 212 may have a function to connect the display module 20 to the support member 50.

Referring to FIGS. 8, 9, and 16, the support member 50 according to an example embodiment may include a second fastening hole 512 corresponding to the hook 211 and the first fastening hole 212. The hook 211 may have a function to connect the display module 20 to the support member 50. The fastening screw inserted into the second fastening hole 512 may have a function to connect the display module 20 to the support member 50.

Referring to FIG. 16, the hook 211 may be connected to a hook catch portion 511. The hook 211 may be hook-coupled to the hook catch portion 511. The second fastening hole 512 may be in contact with the first fastening hole 212. The fastening screw (not shown) may connect the first fastening hole 212 to the second fastening hole 512.

However, the arrangement of the first fastening hole 212 and the hook 211 is not limited to the above description. For example, the hook 211 and the first fastening hole 212 capable of screw coupling may be provided in any one of the display module 20 and the support member 50, and the hook catch portion 511 into which the hook 211 is inserted and the second fastening hole 512 corresponding to the first fastening hole 212 may be provided in the other thereof.

When the guide protrusion 53 of the support member 50 is present in the accommodation position 472 of the fixing member 40, the display module 20 may protrude out of the housing 60. When the display module 20 protrudes out of the housing 60, the fastening screw (not shown) connecting the first fastening hole 212 to the second fastening hole 512 may be easily removed.

The display module 20 according to an example embodiment may include a first electric connection portion 23. The first electric connection portion 23 may include a display control board 22 fixed to the display housing 21. The first electric connection portion 23 may connect a display board 27 supported by a display board support 26.

The support member 50 according to an example embodiment may include a second electric connection portion 54. When the support member 50 is connected to the display module 20, the second electric connection portion 54 may be electrically connected to the first electric connection portion 23. The second electric connection portion 54 may be on a display module connection portion 52 (see FIG. 9) included in the support member 50.

The display module 20 according to an example embodiment may include a display fastening portion 24. The display fastening portion 24 may have a function to fasten the display housing 21, the display board support 26, and the display cover 28, which are included in the display module 20.

FIG. 17 is an enlarged cross-sectional view of a connection portion of the display module, the fixing assembly, and the main body module of the air sterilization module according to various embodiments.

The main body module 70 according to an example embodiment may include a circuit wire 77. One end portion of the circuit wire 77 may be connected to the second electric connection portion 54 included in the support member 50. The other end portion of the circuit wire 77 may be connected to a main body module control board 78 (see FIG. 2) included in the main body module 70. The circuit wire 77 may have a length such that, even when the guide protrusion 53 of the support member 50 is located in the accommodation groove 471 of the fixing member 40, both end portions of the circuit wire 77 may be connected to the second electric connection portion 54 and the main body module control board 78.

The display module 20 may include the display control board 22 on the display housing 21. The display control board 22 may serve to electrically control the display module 20. The display control board 22 may include the first electric connection portion 23. The first electric connection portion 23 may be electrically connected to the second electric connection portion 54.

The display module 20 may include the display board support 26. The display board support 26 may have a function to support the display board 27 included in the display module 20.

Referring to FIGS. 15, 16 and 17 (which may be referred to as FIGS. 15 to 17), the display housing 21 may include the display fastening portion 24. As a fastening screw is inserted through the display fastening portion 24, the display housing 21, the display board support 26, and the display cover 28 may be coupled to one another.

Referring back to FIGS. 16 and 17, the display control board 22 may be electrically connected to the display board 27. The display cover 28 may be electrically connected to the display board 27. Even when the guide protrusion 53 (see FIG. 13) is located in the accommodation groove 471 (see FIG. 13), the display cover 28 and the display board 27 may be electrically connected to each other.

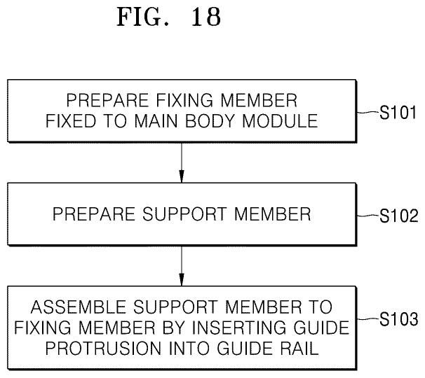

FIG. 18 is a flowchart illustrating an example method of assembling an air sterilization module, according to various embodiments.

Referring to FIG. 18, a method of assembling the air sterilization module 1 according to an example embodiment may include an operation of preparing the fixing member 40 fixed to the main body module 70 (S101). The fixing member 40 may have the guide rail 43.

The method of assembling the air sterilization module 1 according to an example embodiment may include an operation of preparing the support member 50 (S102). The support member 50 may include the guide protrusion 53.

The method of assembling the air sterilization module 1 according to an example embodiment may include an operation of assembling the support member 50 to the fixing member 40 by inserting the guide protrusion 53 into the guide rail 43 (S103).

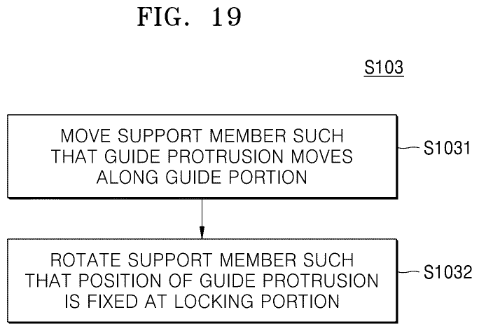

FIG. 19 is a flowchart illustrating an example operation of assembling the support member to the fixing member (S103), according to various embodiments.

Referring to FIG. 19, the operation of assembling the support member 50 to the fixing member 40 (S103) may include an operation of moving the support member 50 such that the guide protrusion 53 moves along the guide portion 44 (S1031).

An operation of assembling the support member 50 to the fixing member 40 (S103) may include an operation of rotating the support member 50 such that the position of the guide protrusion 53 is fixed at the locking portion 45 (S1032).

In the following description, redundant descriptions are omitted, and differences are mainly described.

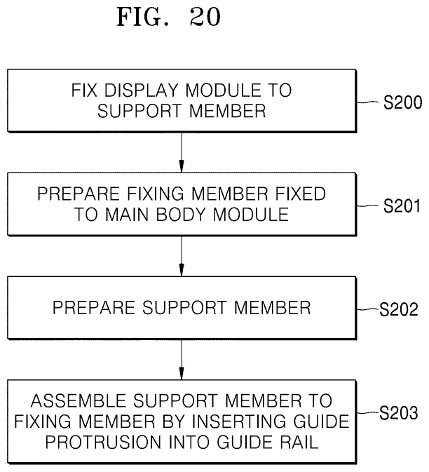

FIG. 20 is a flowchart illustrating an example method of assembling the air sterilization module, according to various embodiments.

Referring to FIG. 20, before the operation of preparing the fixing member 40 fixed to the main body module 70, the method may include an operation of fixing the display module 20 to the support member 50 (S200).

An operation of preparing the fixing member 40 fixed to the main body module 70 (S201), an operation of preparing the support member 50 (S202), and an operation of assembling the support member 50 to the fixing member 40 (S203) may be substantially the same as the previously described operations S101 to S103, respectively.

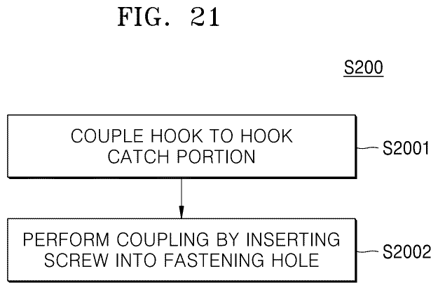

FIG. 21 is a flowchart illustrating an example operation of fixing the display module to the support member (S200), according to various embodiments.

Referring to FIG. 21, the operation of fixing the display module 20 to the support member 50 (S200) may include an operation of hook-coupling the hook 211 of the display module 20 to the hook catch portion 511 of the support member 50 (S2001) and an operation of performing screw-coupling by inserting a fastening screw into the first fastening hole 212 of the display module 20 and the second fastening hole 512 of the support member 50 (S2002).

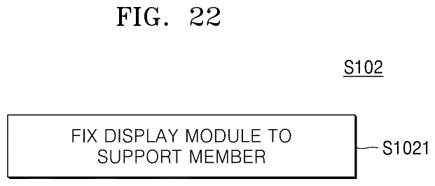

FIG. 22 is a flowchart illustrating an example operation of preparing the support member (S102), according to various embodiments.

Referring to FIG. 22, the operation of preparing the support member 50 (S102) may include an operation of fixing the display module 20 to the support member 50 (S1021).

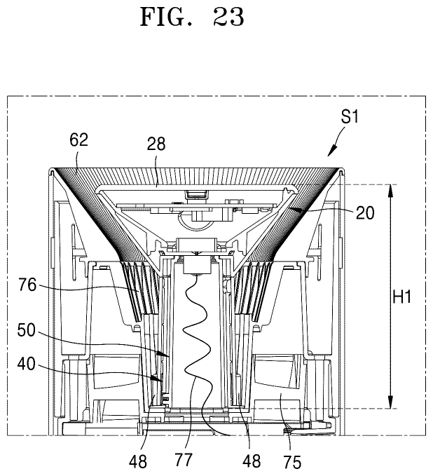

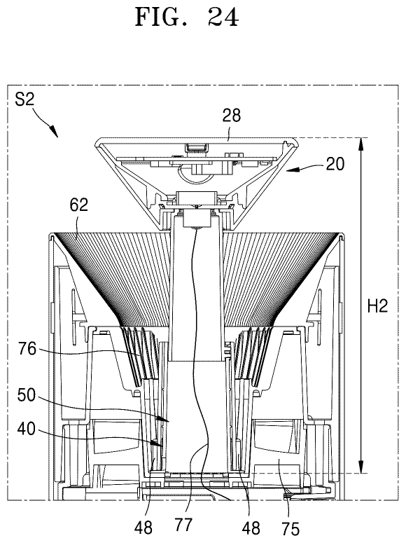

FIG. 23 is a cross-sectional view illustrating an example state of the display module and the fixing assembly, according to various embodiments. FIG. 24 is a cross-sectional view illustrating an example state of the display module and the fixing assembly, according to various embodiments.

Referring to FIGS. 11 and 23, the display module 20 and the fixing assembly 30 according to an example embodiment may have a first state S1 when the guide protrusion 53 of the support member 50 is located at the locking portion 45 of the fixing member 40. The first state S1 may be a state in which a user operates the air sterilization module 1.

A height from a lower surface of the fixing member 40 to the display cover 28 in the first state S1 may be a first height H1.

Referring to FIGS. 13 and 24, the display module 20 and the fixing assembly 30 according to an example embodiment may have a second state S2 when the guide protrusion 53 of the support member 50 is located at the accommodation portion 47 of the fixing member 40. The second state S2 may be a state in which a user separates the display module 20 from the air sterilization module 1.

A height from the lower surface of the fixing member 40 to the display cover 28 in the second state S2 may be a second height H2. The second height H2 may be greater than the first height H1.

Referring to FIGS. 19, 23, and 24, the first state S1 may be changed to the second state S2 via the operation of preparing the fixing member 40 fixed to the main body module 70 (S101), the operation of preparing the support member 50 (S102), and the operation of assembling the support member 50 to the fixing member 40 by inserting the guide protrusion 53 into the guide rail 43 (S103). In this state, rotational power may be applied to the display module 20 relative to the fixing member 40.

Meanwhile, in an example embodiment, an example in which the display module 20 and the fixing assembly 30 for fixing the same to the main body module 70 are applied to the air sterilization module 1 is mainly described. However, application subjects of the display module 20 and the fixing assembly 30 according to an embodiment are not limited thereto, and may be applied to an air purification device 10. The air purification device 10 may include an air purification module 80.

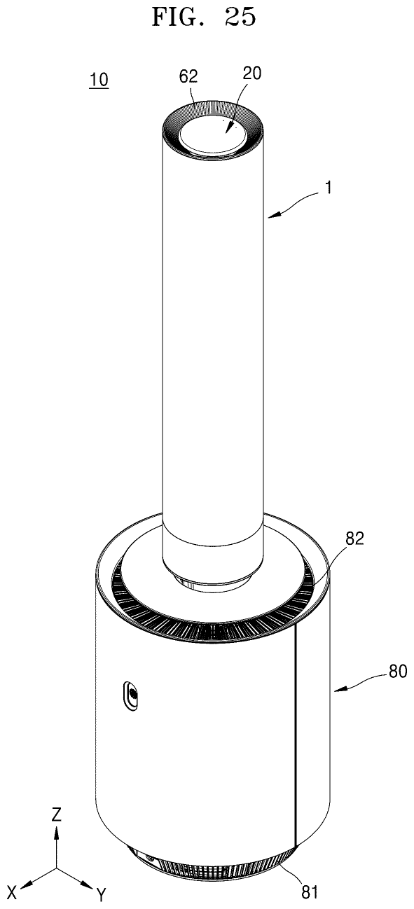

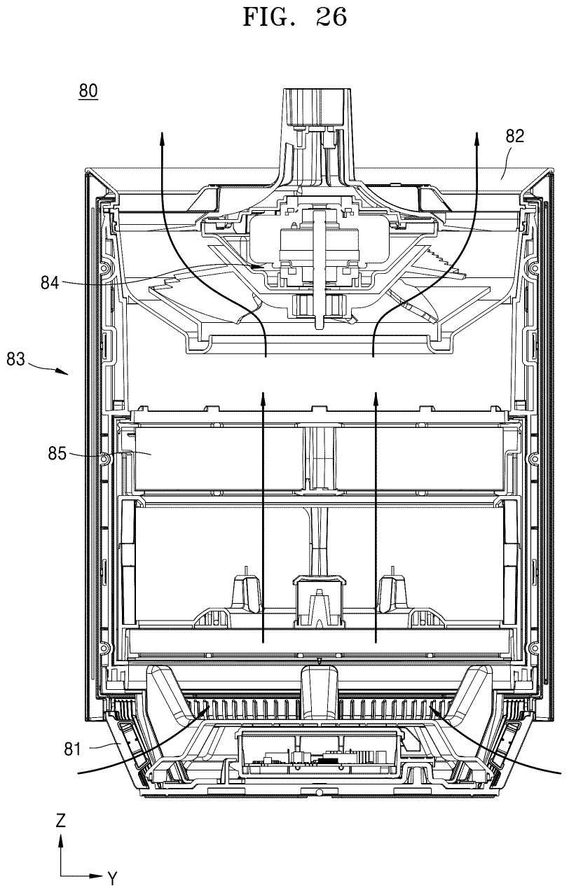

FIG. 25 is a perspective view of the air purification device according to various embodiments. FIG. 26 is a cross-sectional view of the air purification module according to various embodiments.

Referring to FIGS. 25 and 26, the air purification device 10 according to an example embodiment may include the air sterilization module 1 and the air purification module 80. The air sterilization module 1 may sterilize microorganisms in the air. The air purification module 80 may remove particulate from the air.

The air sterilization module 1 may be located above the air purification module 80. However, the disclosure is not limited thereto.

The air purification module 80 may include an air purification intake port 81 and an air purification exhaust port 82. An air purification fan 84 of the air purification module 80 sucks external air through the air purification intake port 81, particulate in the air may be removed through an air purification filter 85, and then, the air removed of particulate may be discharged through the air purification exhaust port 82. The air purification intake port 81 may be located in a lower portion of the air purification module 80, and the air purification exhaust port 82 may be located in an upper portion of the air purification module 80. However, the disclosure is not limited thereto.

Part of exhaust air discharged by the air purification module 80 through the air purification exhaust port 82 may be moved to the air sterilization module 1. Part of the exhaust air discharged through the air purification exhaust port 82 may be sterilized in the air sterilization module 1 and discharged through the exhaust port 62.

The air purification device 10 may include the housings 60 and 83, the main body module 70, the display module 20, and the fixing assembly 30 (see FIG. 6).

The housings 60 and 83 may include the housing 60 of the air sterilization module 1 and the housing 83 of the air purification module 80. The main body module 70 may include the fan 71 of the air sterilization module 1 and the air purification fan 84 of the air purification module 80.

The display module 20 may be fixed to the air purification device 10. The exhaust port 62 may surround the display module 20. The fixing of the display module 20 may be released by holding and rotating an upper end of the display module 20. The display module 20 may be raised above the air purification device 10 by holding and lifting the upper end of the display module 20. The display module 20 may be temporarily fixed by holding and rotating the upper end of the display module 20 raised above the air purification device 10. When the display module 20 is raised above the air purification device 10 and temporarily fixed, the display module 20 may be easily separated from the air purification device 10.

The display module 20 and the fixing assembly 30 according to an example embodiment may provide a user with easy disassembly and assembly of a display assembly.

The display module 20 and the fixing assembly 30 according to an example embodiment may increase manufacturing efficiency of the air sterilization module 1 and facilitate repairs in case of defects.

While the disclosure has been particularly shown and described with reference to various example embodiments using specific terminologies, the various embodiments and terminologies should be considered in descriptive sense only and not for purposes of limitation. Therefore, it will be understood by those of ordinary skill in the art that various changes in form and detail may be made therein without departing from the spirit and scope of the disclosure including the appended claims and their equivalents. It will also be understood that any of the embodiment(s) described herein may be used in conjunction with any other embodiment(s) described herein.

An air sterilization module according to an example embodiment includes: a housing including an intake port and an exhaust port, a main body module provided inside the housing and including a fan configured to generate airflow, a display module including a display provided to be exposed to outside of the housing, and a fixing assembly comprising a fastener separably fixing the display module to the main body module, wherein the fixing assembly may include a fixing member comprising a guide rail or protrusion fixed to the main body module and a support member comprising a support configured to be assembled to the fixing member while supporting the display module, any one of the fixing member and the support member may include a guide rail extending in a direction in which the support member is assembled, and an other of the fixing member and the support member may include a guide protrusion inserted into the guide rail and movable along the guide rail.

The guide rail may include a plurality of guide rails provided on the fixing member, and the guide protrusion may include a plurality of guide protrusions provided on the support member.

The guide rail may include a guide portion extending in a first direction such that the guide protrusion is movable in a longitudinal direction of the housing, and a locking portion extending from the guide portion in a second direction intersecting the first direction and configured to fix a position of the guide protrusion, such that the support member is rotated and fixed to the fixing member.

The guide rail may include an accommodation portion extending from the guide portion in a third direction intersecting the first direction, and configured to accommodate the guide protrusion in a process of assembling the support member to the fixing member, and an accommodation groove may be provided in the accommodation portion, the accommodation groove restricting a movement of the guide protrusion when the guide protrusion is located in the accommodation portion.

The guide protrusion may include a plurality of partial protrusions that are elastically deformable in a direction perpendicular to the direction in which the support member is assembled.

A first fastening hole and a hook capable of screw-coupling may be provided in any one of the display module and the support member, a hook catch portion into which the hook is inserted and a second fastening hole corresponding to the first fastening hole may be provided in the other of the display module and the support member, the display module and the support member may be hook-coupled to each other by the hook and the hook catch portion, and a fastening screw is screw-coupled by being inserted into the first fastening hole and the second fastening hole.

The display module may be arranged above the housing.

The main body module may further include an upper light blocking member comprising a light blocking material provided inside the housing, and the fixing member is fixed to the upper light blocking member.

The display module may include a first electric connection portion, the main body module may further include a circuit wire having one end portion electrically connected to the first electric connection portion, and the length of the circuit wire may be equal to or greater than the length in which an electrical signal is transmittable to the first electric connection portion when at least the guide protrusion is located in the accommodation portion.

The fixing assembly may further include a guide protection case arranged to surround the fixing member to prevent or block the guide rail and the guide protrusion inserted into the guide rail from being exposed to outside.

An air purification device according to an example embodiment includes: a housing including an intake port and an exhaust port, a main body module provided inside the housing and including a fan that configured to generate airflow, a display module comprising a display provided to be exposed to outside of the housing, and a fixing assembly comprising a guide rail or guide protrusion configured to separably fix the display module to the main body module, wherein the fixing assembly may include a fixing member fixed to the main body module and a support member comprising a support configured to be assembled to the fixing member while supporting the display module, any one of the fixing member and the support member may include a guide rail extending in a direction in which the support member is assembled, and an other of the fixing member and the support member may include a guide protrusion capable of moving along the guide rail.

The display module may have a first state based on the guide protrusion being located at one end portion of the guide rail, and a second state based on the guide protrusion being located at the other end portion of the guide rail.

The first state may be changed to the second state by applying rotational power to the display module relative to the fixing member.

A method of assembling an air sterilization module according to an example embodiment, wherein the air sterilization module includes: a air sterilization module including: a housing, a main body module comprising a fan provided inside the housing, a display module comprising a display provided to be exposed to outside of the housing, and a fixing assembly comprising a fastener separably fixing the display module to the main body module, the method including: preparing a fixing member fixed to the main body module and including a guide rail extending in an assembly direction, preparing a support member comprising a support on which a guide protrusion insertable into a guide rail is provided, and assembling the support member to the fixing member by inserting the guide protrusion into the guide rail, wherein the guide rail may include: a guide portion extending in a first direction such that the guide protrusion is movable in a longitudinal direction of the housing, and a locking portion extending from the guide portion in a second direction intersecting the first direction and configured to fix a position of the guide protrusion, and the assembling the support member to the fixing member may include moving the support member such that the guide protrusion moves along the guide portion, and rotating the support member such that the position of the guide protrusion is fixed at the locking portion.

The assembling the support member to the fixing member or the operation of preparing the support member may further include fixing the display module to the support member and the fixing the display module to the support member including an operation of hook-coupling a hook of the display module to a hook catch portion of the support member, and performing screw-coupling by inserting a fastening screw into a first fastening hole of the display module and a second fastening hole of the support member.

What is claimed is:

1. An air sterilization module comprising:
a housing comprising an intake port and an exhaust port;
a main body module provided inside the housing and comprising a fan configured to generate airflow;
a display module comprising a display configured to be exposed to outside of the housing; and
a fixing assembly comprising a fastener configured to separably fix the display module to the main body module,
wherein the fixing assembly comprises a fixing member comprising a guide rail or protrusion fixed to the main body module and a support member comprising a support configured to be assembled to the fixing member while supporting the display module,
any one of the fixing member and the support member comprises a guide rail extending in a direction in which the support member is assembled, and
an other of the fixing member and the support member comprises a guide protrusion inserted into the guide rail and movable along the guide rail.

2. The air sterilization module of claim 1, wherein
the guide rail comprises a plurality of guide rails provided on the fixing member, and
the guide protrusion comprises a plurality of guide protrusions provided on the support member.

3. The air sterilization module of claim 1, wherein
the guide rail comprises:
a guide portion extending in a first direction such that the guide protrusion is movable in a longitudinal direction of the housing; and
a locking portion extending from the guide portion in a second direction intersecting the first direction and configured to fix a position of the guide protrusion, such that the support member is rotated and fixed to the fixing member.

4. The air sterilization module of claim 3, wherein
the guide rail comprises
an accommodation portion extending from the guide portion in a third direction intersecting the first direction, and configured to accommodate the guide protrusion in a process of assembling the support member to the fixing member, and
an accommodation groove provided in the accommodation portion, the accommodation groove configured to restrict a movement of the guide protrusion based on the guide protrusion being located in the accommodation portion.

5. The air sterilization module of claim 3, wherein
the guide protrusion comprises
a plurality of partial protrusions that are elastically deformable.

6. The air sterilization module of claim 1, wherein
a first fastening hole and a hook capable of screw-coupling are provided in any one of the display module and the support member,
a hook catch portion into which the hook is insertable and a second fastening hole corresponding to the first fastening hole are provided in the other of the display module and the support member,
the display module and the support member are hook-coupled to each other by the hook and the hook catch portion, and a fastening screw is screw-coupled by being inserted into the first fastening hole and the second fastening hole.

7. The air sterilization module of claim 1, wherein
the display module is arranged above the housing.

8. The air sterilization module of claim 1, wherein
the main body module further comprises an upper light blocking member comprising a light blocking material provided inside the housing, and
the fixing member is fixed to the upper light blocking member.

9. The air sterilization module of claim 4, wherein
the display module comprises a first electric connection portion,
the main body module further comprises a circuit wire having one end portion electrically connected to the first electric connection portion, and
a length of the circuit wire is equal to or greater than a length in which an electrical signal is transmittable to the first electric connection portion based on at least the guide protrusion being located in the accommodation portion.

10. The air sterilization module of claim 4, wherein
the fixing assembly further comprises
a guide protection case arranged to surround the fixing member and configured to block the guide rail and the guide protrusion inserted into the guide rail from being exposed to outside.

11. An air purification device comprising: a housing comprising an intake port and an exhaust port;
a main body module provided inside the housing and comprising a fan configured to generate airflow;
a display module comprising a display configured to be exposed to outside of the housing; and
a fixing assembly comprising a fastener configured to separably fix the display module to the main body module,
wherein the fixing assembly comprises a fixing member comprising a guide rail or protrusion fixed to the main body module and a support member comprising a support configured to be assembled to the fixing member while supporting the display module,
any one of the fixing member and the support member comprises a guide rail extending in a direction in which the support member is assembled, and
an other of the fixing member and the support member comprises a guide protrusion capable of moving along the guide rail.

12. A method of assembling an air sterilization module, the air sterilization module comprising: a housing, a main body module provided inside the housing, a display module provided to be exposed to outside of the housing, and a fixing assembly separably fixing the display module to the main body module, the method comprising:
preparing a fixing member fixed to the main body module and comprising a guide rail extending in an assembly direction;
preparing a support member on which a guide protrusion insertable into a guide rail is provided; and
assembling the support member to the fixing member by inserting the guide protrusion into the guide rail,
wherein the guide rail comprises: a guide portion extending in a first direction such that the guide protrusion is movable in a longitudinal direction of the housing; and a locking portion extending from the guide portion in a second direction intersecting the first direction and configured to fix a position of the guide protrusion, and
the assembling the support member to the fixing member comprises:
moving the support member such that the guide protrusion moves along the guide portion; and
rotating the support member such that the position of the guide protrusion is fixed at the locking portion.

13. The method of claim 12, wherein
the assembling the support member to the fixing member or the preparing the support member further comprises fixing the display module to the support member,
the fixing the display module to the support member comprising:
hook-coupling a hook of the display module to a hook catch portion of the support member; and
performing screw-coupling by inserting a fastening screw into a first fastening hole of the display module and a second fastening hole of the support member.

14. The method of claim 12, wherein
the display module has an assembly ready state based on the guide protrusion being located at the guide portion, and
a locking state based on the support member being fixed at the locking portion.

15. The method of claim 12, wherein,
by applying rotational power to the display module relative to the fixing member, a locking state is changed to an assembly ready state.
